(19) United States
(12) Patent Application Publication
(10) Pub. No.: US 2008/0096846 A1
(43) Pub. Date: Apr. 24, 2008

(54) Arene Ruthenium (ll) Compounds And Their Use In Cancer Therapy

(76) Inventors: Abraha Habtemariam, Edinburgh (GB); Peter John Sadler, Edinburgh (GB)

Correspondence Address: CURATOLO SIDOTI CO., LPA, 24500 CENTER RIDGE ROAD, SUITE 280, CLEVELAND, OH, 44145, US

(21) Appl. No.: 11/660,627

(22) PCT Filed: Aug. 19, 2005
(86) PCT No.: PCT/GB05/03242

§ 371 Date: Feb. 20, 2007

(30) Foreign Application Priority Data
Aug. 20, 2004 (GB) 0418613.3

(51) Int. Cl. A61K 31/28; A61K 31/555; A61K 31/655; A61P 35/00; C07C 245/00; C07F 15/00

(52) U.S. Cl. 514/150; 514/188; 514/492; 534/550; 546/4; 556/137; 556/37

(57) ABSTRACT

A ruthenium (II) compound of formula (I): or a solvate on prodrug thereof, wherein: R¹, R¹, R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings; X is a neutral or negatively charged N- or S-donor ligand; Y is a counterion; in is 0 or 1; q is 1, 2 or 3; C″ is C₁₋₁₂ alkylene bound to two A groups; p is 0 or 1 and r is 1 when p is 0 and r is 2 when p is 1; and A and B are each independently O-donor, N-donor or S-donor ligands, and may be connected to one another. The compounds are used in cancer therapy.

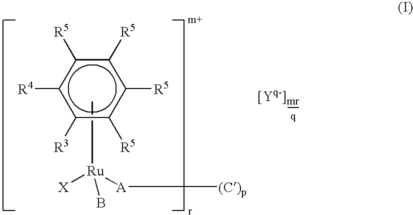

This invention relates to ruthenium (II) compounds, to their use in medicine, particularly for the treatment and/or prevention of cancer, and to a process for their preparation.

WO 01/30790 and WO 02/02572 disclose ruthenium (II) compounds for use in the treatment of cancer. These compounds can be described as half-sandwich compounds, having an arene ring bound to the ruthenium, as well as other non-arene ligands. The compounds exemplified in these applications have as one of the ligands a halo atom. Without wishing to be bound by theory, it is thought that the hydrolysis of the halo atom activates the complexes and allows them to bind to DNA.

The present inventors have studied the hydrolysis rates of a number of different ligands including halo and have surprisingly found that complexes containing ligands that have longer hydrolysis times still exhibit anti-tumour activity.

According to the present invention there is provided a ruthenium (II) compound of formula (I): 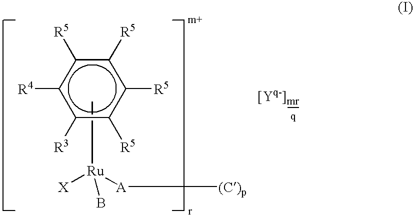
or a solvate or prodrug thereof, wherein:
R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is a neutral or negatively charged N- or S-donor ligand;
Y is a counterion;
m is 0 or 1;
q is 1, 2 or 3;
C′ is C₁₋₁₂ alkylene bound to two A groups;
p is 0 or 1 and r is 1 when p is 0 and r is 2 when p is 1; and
A and B are each independently O-donor, N-donor or S-donor ligands.

When p is 1, the ligand A is bound to another ligand A such that the compound comprises two ruthenium atoms. Such complexes are called dinuclear complexes.

Ligands A and B may be connected to one another, but they cannot be bound to ligand X.

A second aspect of the present invention provides a composition comprising a compound of the first aspect and a pharmaceutically acceptable carrier or diluent.

A third aspect of the invention provides the use of a compound of the first aspect in a method of therapy.

A fourth aspect of the invention provides the use of a compound of the first aspect in the preparation of a medicament for the treatment of cancer.

A fifth aspect of the invention provides a method of treatment of a subject suffering from cancer, comprising administering to such a subject a therapeutically-effective amount of a compound of the first aspect, preferably in the form of a pharmaceutical composition.

DEFINITIONS

N-donor ligands: N-donor ligands are ligands which bind to a metal atom via a nitrogen atom. They are well known in the art and include: nitrile ligands (N≡C—R); azo ligands (N═N—R); aromatic N-donor ligands; amine ligands (NR^{N1}R^{N2}R^{N3}); azide (N₃⁻); cyanide (N≡C⁻); isothiocyanate (NCS⁻).

In both nitrile and azo ligands R may be selected from C₁₋₇ alkyl and C₅₋₂₀ aryl.

Aromatic N-donor ligands include optionally substituted pyridine, pyridazine, pyrimidine, purine and pyrazine. The optional substituents may be selected from cyano, halo and C₁₋₇ alkyl.

R^{N1}, R^{N2} and R^{N3} may be independently selected from H and C₁₋₇ alkyl, or if A and B are both amine ligands, R^{N1} on each ligand join together to form a C₁₋₇ alkylene chain.

When p is 1, R^{N2} on each A ligand join together form the group C′ which is C₁₋₁₂ alkylene.

S-donor ligands: S-donor ligands are ligands which bind to a metal atom via a sulphur atom. They are well known in the art and include: thiosulfate (S₂O₃²⁻); isothiocyanate (NCS⁻); thiocyanate (CNS⁻); sulfoxide ligands (R^{S1}R^{S2}SO); thioether ligands (R^{S1}R^{S2}S); thiolate ligands (R^{S1}S⁻); sulfinate ligands (R^{S1}SO₂—); and sulfenate ligands (R^{S1}SO⁻), wherein R^{S1} and R^{S2} are independently selected from C₁₋₇ alkyl and C₅₋₂₀ aryl, which groups may be optionally substituted.

O-donor ligands: O-donor ligands are ligands which bind to a metal atom via an oxygen atom. They are well known in the art and include: carbonate (CO₃⁻); carboxylate ligands (R^{C}CO₂⁻); nitrate (NO₃⁻); sulfate (SO₄²⁻) and sulphonate (R^{S1}O₃⁻), wherein R^{C} is selected from C₁₋₇ alkyl and C₅₋₂₀ aryl and R^{S1} is as defined above.

C₁₋₇ Alkyl: The term “C₁₋₇ alkyl”, as used herein, pertains to a monovalent moiety obtained by removing a hydrogen atom from a carbon atom of a hydrocarbon compound having from 1 to 7 carbon atoms, which may be aliphatic or alicyclic, and which may be saturated or unsaturated (e.g., partially unsaturated, fully unsaturated). Thus, the term “alkyl” includes the sub-classes alkenyl, alkynyl, cycloalkyl, cycloalkyenyl, cylcoalkynyl, etc., discussed below.

Examples of saturated C₁₋₇ alkyl groups include, but are not limited to, methyl (C₁), ethyl (C₂), propyl (C₃), butyl (C₄), pentyl (C₅), hexyl (C₆) and heptyl (C₇).

Examples of saturated linear C₁₋₇ alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of saturated branched C₁₋₇ alkyl groups include iso-propyl (C₃), iso-butyl (C₄), sec-butyl (C₄), tert-butyl (C₄), iso-pentyl (C₅), and neo-pentyl (C₅).

C₂₋₇ Alkenyl: The term “C₂₋₇ alkenyl”, as used herein, pertains to an alkyl group having one or more carbon-carbon double bonds. Examples of C₂₋₇ alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₇ Alkynyl: The term “C₂₋₇ alkynyl”, as used herein, pertains to an alkyl group having one or more carbon-carbon triple bonds. Examples of C₂₋₇ alkynyl groups include, but are not limited to, ethynyl (ethinyl, —C═CH) and 2-propynyl (propargyl, —CH₂—C≡CH).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g., partially unsaturated, fully unsaturated), which moiety has from 3 to 7 carbon atoms. Thus, the term “C₃₋₇ cycloalkyl” includes the sub-classes cycloalkyenyl and cycloalkynyl. Examples of cycloalkyl groups include, but are not limited to, those derived from:

saturated hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇); and

unsaturated hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇).

The alkyl groups in the compounds of the invention may optionally be substituted. Substituents include one or more further alkyl groups and/or one or more further substituents, such as, for example, C₅₋₂₀ aryl (e.g. benzyl), C₃₋₂₀ heterocyclyl, cyano (—CN), nitro (—NO₂), hydroxyl (—OH), ester, halo, thiol (—SH), thioether and sulfonate (—S(═O)₂)OR, where R is wherein R is a sulfonate substituent, for example, a C₁₋₇ alkyl group, a C₃₋₂₀heterocyclyl group, or a C₅₋₂₀aryl group, preferably a C₁₋₇ alkyl group).

C₂₋₁₂ alkylene: The term “C₂₋₁₂ alkylene” is defined similarly to the definition of the term “alkyl” but includes C₂ to C₁₂ groups and is a divalent species with radicals separated by two or more (e.g. from two to twelve) carbon atoms linked in a chain. Preferably, the alkylene groups are straight chain groups. C₂₋₁₂ alkylene groups are optionally substituted in the alkylene chain, preferably with one or more phenylene (e.g., 1-4-phenylene) and/or —CONR^{1a}— groups and/or —NR^{2a}— groups, where R^{1a} and R^{2a} independently represent H, C₁₋₇ alkyl, C₃₋₂₀ heterocyclyl or C₅₋₂₀ aryl. Preferably, R^{1a} and R^{2a} are H or C₁ to C₃ alkyl.

C₅₋₂₀ Aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₆ aryl”, as used herein, pertains to an aryl group having 5 or 6 ring atoms.

The ring atoms may be all carbon atoms, as in “carboaryl groups”. Examples of carboaryl groups include C₃₋₂₀ carboaryl, C₅₋₂₀ carboaryl, C₅₋₁₅ carboaryl, C₅₋₁₂ carboaryl, C₅₋₁₀ carboaryl, C₅₋₇ carboaryl, C₅₋₆ carboaryl, C₅ carboaryl, and C₆ carboaryl.

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of heteroaryl groups include C₃₋₂₀ heteroaryl, C₅₋₂₀ heteroaryl, C₅₋₁₅ heteroaryl, C₅₋₁₂ heteroaryl, C₅₋₁₀ heteroaryl, C₅₋₇ heteroaryl, C₅₋₆ heteroaryl, C₅ heteroaryl, and CG heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl groups which comprise fused rings, include, but are not limited to:

C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ heteroaryl groups (with 2 fused rings) derived from benzodiazepine (N₂);

C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

C₅₋₂₀ aryl groups may optionally be substituted with one or more substituents including, for example, C₁₋₇ alkyl, C₅₋₂₀ aryl, C₃₋₂₀ heterocyclyl, cyano, nitro, hydroxyl, ester, halo, thiol, thioether and sulfonate.

C₃₋₂₀ Heterocyclyl: The term “C₃₋₂₀ heterocyclyl”, as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₅);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and

N₁O₁S₁: oxathiazine (C₆).

C₃₋₂₀ heterocyclyl groups may optionally be substituted with one or more substituents including, for example, C₁₋₇ alkyl, C₅₋₂₀ aryl, C₃₋₂₀ heterocyclyl, cyano, nitro, hydroxyl, ester, halo, thiol, thioether and sulfonate.

Halo: —F, —Cl, —Br, and —I.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, —NHCH₂Ph and —NHPh.

Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Sulfo: —S(═O)₂OH, —SO₃H.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Azo: —N═N—R, where R is an azo substituent, for example a C₁₋₇ alkyl group, a C₃₋₂₀ heterocyclyl group, or a C₅₋₂₀ aryl group, preferably a C₁₋₇ alkyl group. Examples of azo groups include, but are not limited to, —N═N—CH₃ and —N═N-Ph.

Heterocyclic ring: The term “heterocyclic ring” as used herein refers to a 3-, 4-, 5-, 6-, 7-, or 8- (preferably 5-, 6- or 7-) membered saturated or unsaturated ring, which may be aromatic or non-aromatic, containing from one to three heteroatoms independently selected from N, O and S, e.g. indole (also see above).

Carbocyclic ring: The term “carbocyclic ring” as used herein refers to a saturated or unsaturated ring, which may be aromatic or non-aromatic, containing from 3 to 8 carbon atoms (preferably 5 to 7 carbon atoms) and includes, for example, cyclopropane, cyclobutane, cyclopentane, cyclohexane and cycloheptane (also see above).

Includes Other Forms

Unless otherwise specified, included in the above are the well known ionic, solvate, and protected forms of these substituents. For example, a reference to carboxylic acid (—COOH) also includes the anionic (carboxylate) form (—COO⁻) or solvate thereof, as well as conventional protected forms. Similarly, a reference to an amino group includes the protonated form (—N⁺HR¹R²) or solvate of the amino group, as well as conventional protected forms of an amino group. Similarly, a reference to a hydroxyl group also includes the anionic form (—O⁻) or solvate thereof, as well as conventional protected forms.

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: ketolenol (illustrated below), imine/enamine, amide/imino alcohol, amidine/amidine, nitroso/oxime, thioketone/enethiol, N-nitroso/hydroxyazo, and nitro/aci-nitro.

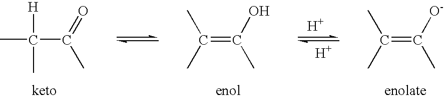

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof, for example, a mixture enriched in one enantiomer. Methods for the preparation (e.g., asymmetric synthesis) and separation (e.g., fractional crystallisation and chromatographic means) of such isomeric forms are either known in the art or are readily obtained by adapting the methods-taught herein, or known methods, in a known manner.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄CrH₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O—).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also include prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:
C₁₋₇alkyl
(e.g., -Me, -Et, -nPr, -iPr, -nBu, -sBu, -iBu, -tBu);
C₁₋₇aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Use of Compounds of the Invention

The invention provides compounds of formula (I), or solvates or prodrugs thereof (“active compounds”), for use in a method of treatment of the human or animal body. Such a method may comprise administering to such a subject a therapeutically-effective amount of an active compound, preferably in the form of a pharmaceutical composition.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of an active compound, or a material, composition or dosage form comprising an active compound, which is effective for producing some desired therapeutic effect, commensurate with a reasonable benefit/risk ratio.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insulation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

Cancers

Examples of cancers which may be treated by the active compounds include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

Examples of other therapeutic agents that may be administered together (whether concurrently or at different time intervals) with the compounds of the formula (I) include but are not limited to topoisomerase inhibitors, alkylating agents, antimetabolites, DNA binders and microtubule inhibitors (tubulin target agents), such as cisplatin, cyclophosphamide, doxorubicin, irinotecan, fludarabine, 5FU, taxanes, mitomycin C or radiotherapy. For the case of active compounds combined with other therapies the two or more treatments may be given in individually varying dose schedules and via different routes.

The combination of the agents listed above with a compound of the present invention would be at the discretion of the physician who would select dosages using his common general knowledge and dosing regimens known to a skilled practitioner.

Where the compound of the formula (I) is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

Preferences

R¹-R⁶

In one group of embodiments of the present invention, R¹ and R² together with the ring to which they are attached form a saturated or unsaturated carbocyclic or heterocyclic group containing up to 3- to 8-membered carbocyclic or heterocyclic rings, wherein each carbocyclic or heterocyclic ring may be fused to one or more other carbocyclic or heterocyclic rings.

In this group of embodiments, it is preferred that R³, R⁴, R⁵ and R⁶ are H.

R¹ and R² together with the ring to which they are bound in compounds of formula (I) may represent an ortho- or peri-fused carbocyclic or heterocyclic ring system.

R¹ and R² together with the ring to which they are bound may represent a wholly carbocyclic fused ring system such as a ring system containing 2 or 3 fused carbocyclic rings, e.g. optionally substituted, optionally hydrogenated naphthalene or anthracene.

Alternatively, R¹ and R² together with the ring to which they are bound in compounds of formula (I) may represent a fused tricyclic ring such as anthracene or a mono, di, tri, tetra or higher hydrogenated derivative of anthracene. For example, R¹ and R² together with the ring to which they are bound in formula (I) may represent anthracene, 1,4-dihydroanthracene or 1,4,9,10-tetrahydroanthracene.

R¹ and R² together with the ring to which they are bound in formula (I) may also represent:

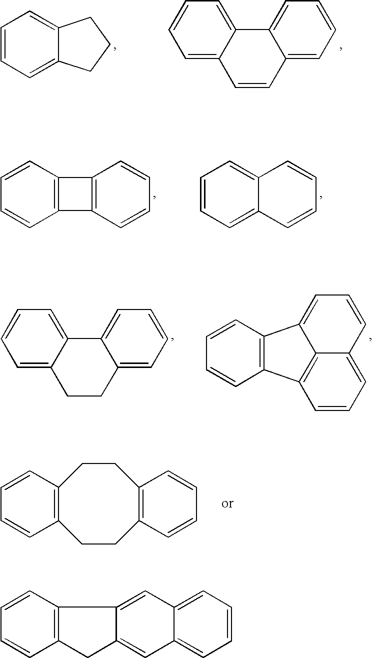

In another group of embodiments, R¹, R², R³, R⁴, R⁵ and R⁶ are independently selected from H, C₁₋₇ alkyl, C₅₋₂₀aryl, C₃₋₂₀ heterocyclyl, halo, ester, amido, acyl, sulfo, sulfonamido, ether, thioether, azo and amino. In this group of embodiments, R¹, R², R³, R⁴, R⁵ and R⁶ are preferably independently selected from H, C₁₋₇ alkyl, C₅₋₂₀ aryl and ester. Of these H and C₁₋₇ alkyl (in particular C₁₋₃ alkyl) are most preferred.

In this group of embodiments, four, five or six of R¹, R², R³, R⁴, R⁵ and R⁶ are preferably hydrogen, with the other (if any) groups being selected from C₁₋₇ alkyl, C₅₋₂₀aryl, C₃₋₂₀ heterocyclyl, halo, ester, amido, acyl, sulfo, sulfonamido, ether, thioether, azo and amino, or more preferably C₁₋₇ alkyl, C₅₋₂₀ aryl and ester, and most preferably C₁₋₇ alkyl (in particular C₁₋₃ alkyl). If two of R¹, R², R³, R⁴, R⁵ and R⁶ are not H, then these groups are preferably meta or para to one another, and more preferably para to one another.

Examples of particularly preferred substitutent patterns include, but are not limited to: phenyl; 1-methyl; and 4-iso-propyl.

A and B

It is preferred that A and B together represent NR^{N4}R^{N5}—(CR^{C1}R^{C2})ₙ—NR^{N6}R^{N7}, wherein R^{C1} and R^{C2} are independently selected from H and C₁₋₄ alkyl, R^{N4}, R^{N5}, R^{N6} and R^{N7} are independently selected from H and C₁₋₄ alkyl, and n is an integer from 1 to 4.

Preferably, R¹⁴ and R¹⁵ are both hydrogen. Preferably n is 2 or 3, more preferably 2. R^{N4}, R^{N5}, R^{N6} and R^{N7} are preferably H or methyl and, more preferably, all of R^{N4}, R^{N5}, R^{N6} and R^{N7} are H.

When R^{N4} is present in A, then p is 0. When R^{N4} is absent, then p is I and C′ takes the place of R^{N4}. In a group of embodiments, R^{N4} is absent from A, p is I and preferably C′ is C₄₋₁₀alkylene with no substituents (e.g. hexylene).

Examples of dinuclear complexes of this group of embodiments are those in which pairs of A and B together with linker C′ represent: 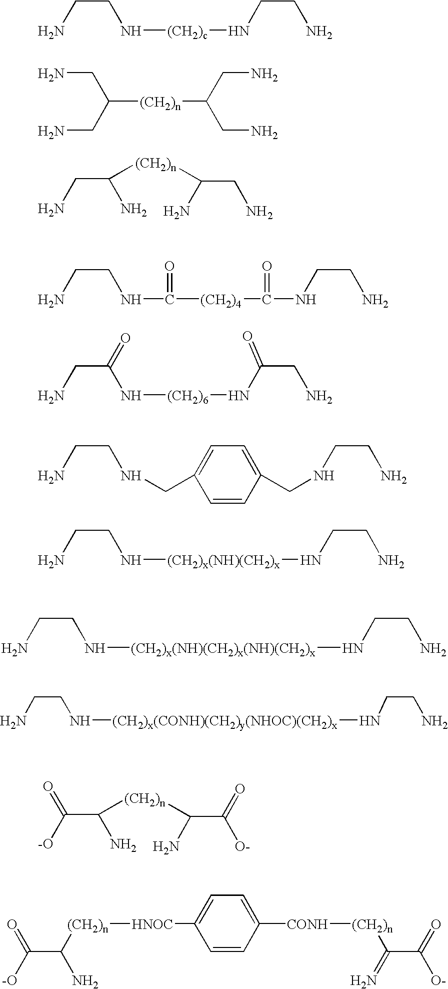
wherein each n′, n″, x′, x″ and y′ independently represents an integer from 1 to 12, preferably 1 to 6.
X

When X is an N-donor ligand, it is preferably selected from azide, isothiocyanate, and optionally substituted pyridine ligands. Of these, azide and isothiocyanate are preferred.

When X is an optionally substituted pyridine ligand, the ligand is preferably at least mono-substituted, and may be di-substituted. These substituents are preferably selected from halo (e.g. chloro, fluoro), cyano, and lower alkyl (e.g. methyl). Of these, chloro, cyano and methyl are preferred. Preferred substituent patterns include, but are not limited to, 3-, 5-dichloro, 4-cyano and 3-methyl.

In some embodiments, X is selected from nitrile ligands (N═C—R); azo ligands (N═N—R); amine ligands (NR^{N1}R^{N2}R^{N3}); azide (N₃⁻); cyanide (N≡C⁻) and isothiocyanate (NCS⁻).

If X is an S-donor ligand, it is preferably a thiolate ligand, for example, PhS⁻.

Y^{q-}

Y^{q-} in compounds of formula (I) is a counterion and is only present in the compound when the complex containing the metal ion is charged. Y^{q-} is preferably a non-nucleophilic anion such as PF₆⁻, BF₄⁻, BPh₄⁻ or CF₃O₂SO⁻, for example.

General Synthesis Methods

The present invention also provides a process for preparing the compounds of the invention which comprises the reaction of a compound of formula [(η⁶-C₆(R¹)(R²)(R³)(R⁴)(R⁵)(R⁶))RuABCl]ⱼ[Y^{q-}], which may be in the form of a monomer or a dimer, with AgNO₃ in a suitable solvent for the reaction, followed by removal of AgCl and reaction with MX, optionally in the presence, or with subsequent addition of, Y^{q-}, in a suitable solvent for the reaction, wherein R¹, R², R³, R⁴, R⁵, R⁶, X, A, B and Y are as defined above for the compounds of the invention, and M is an appropriate cation, e.g. Na⁺.

Preferred reaction conditions include:

(a) stirring the starting ruthenium complex, as described above, in a 1:1 mixture of MeOH and H₂O as a solvent with AgNO₃;

(b) filtering off the AgCl precipitate formed;

(c) adding MX (which may be dissolved by heating, if necessary) and allowing to react;

(d) adding a source of Y^{q-}, such as a compound of formula (NH₄⁺)Y^{q-}, e.g., NH₄ PF₆, and evaporating the filtrate to yield the product.

The filtrate may be purified, for example, by recrystallisation from acetone.

The following non-limiting examples illustrate the present invention.

### EXAMPLES

General Methods

Electrospray Ionisation Mass Spectrometry (ESI-MS): Positive-ion electrospray ionisation mass spectra were obtained with a Platform II mass spectrometer (Micromass, Manchester, U.K.). For offline ESI-MS assays, the samples were prepared in 50% CH₃CN/50% H₂O (v/v) and infused directly into the mass spectrometer at 6 μL min⁻. The ions were produced in an atmospheric pressure ionisation (API)/ESI ion source. For the online LC-ESI-MS assays, a Waters 2690 HPLC system was interfaced with the mass spectrometer, using the same column and gradients as described above for the HPLC assays with a flow rate of 1.0 mL min⁻¹ and a splitting ratio of 1/5. The spray voltage was 3.50-3.68 kV. The cone voltage was varied over the range of 15-30 V as required. The capillary temperature was 338 K for direct infusion and 413 K for the HPLC sampling, with a 450 L h⁻¹ flow of nitrogen drying gas. The quadrupole analyser, operated at a background pressure of 2×10⁻⁵ Torr, was scanned at 300 Da s⁻¹ for direct infusion and 750 Da s⁻¹ for HPLC sampling. Data were collected (for 10 scans during the direct infusion assays) and analysed on a Mass Lynx (ver. 2.3) Windows NT PC data system using the Max Ent Electrospray software algorithm and calibrated versus an NaI calibration file. The mass accuracy of all measurements was within 0.1 m/z unit.

X-ray crystallography: All data were collected at 150 K on a Bruker Smart Apex CCD diffractometer equipped with an Oxford Cryosystems low-temperature device. Following application of a multi-scan absorption correction (SADABS)(Sheldrick, G. M., SADABS, Program for carrying-out multiscan absorption corrections, University of Göttingen, Germany, 1998) the structures were all solved by direct methods (Shelxs, SIR92, Dirdif) (Sheldrick, G. M., SHELXS and SHELXL. Programs for the solution and refinement of crystal structures, University of Göttingen, Germany, 1998; Altomare, A., et al., A. J. Appl. Crystallogr., 26, 343-350 (1993); Beurskens, P. T., et al., The DIRDIF96 Program System, Technical Report of the Crystallography Laboratory, University of Nijrnegen, The Netherlands (1996)) and refined against F² using all data (SHELXL) (Betteridge, P. W., et al., J. Appl. Cryst, 36, 1487 (2003))

### Comparative Example 1

### Synthesis of [(η⁶-C₆H₅C₆H₅)Ru(en)Cl][PF₆] (C1)

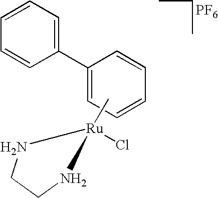

This compound was synthesised as described in Morris, R. E., et al., J. Med. Chem., 44, 3616-3621 (2001)—compound 9.

### Example 1

### Synthesis of [(η⁶-C₆H₅C₆H₅)Ru(en)N₃][PF₆](1)

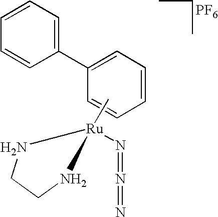

This complex was prepared by refluxing complex C1 (25.0 mg, 0.0496 mmol) and AgNO₃ (8.4 mg, 0.0494 mmol) in 2.5 mL of a 1:1 mixture of MeOH and H₂O for one hour. AgCl was removed by filtration. NaN₃ was added (163 mg, 2.51 mmol), dissolved by heating, and left overnight. NH₄ PF₆ (250 mg) was added, leading to a microcrystalline, yellow precipitate. Recrystallization of the precipitate from acetone gave a yellow crystalline product. Yield of 1: 8.6 mg (34%).

Anal. Calcd for C₁₄F₆H₁₈N₅PRu: C 33.47, H 3.61, N 13.94. Found: C 33.37, H 3.46, N 13.68. MS: m/z 357.7 for [M-PF₆]⁺ (calc. 357.1)

### Comparative Example 2

### Synthesis of [(η⁶-C₆(CH₃)₆)Ru(en)Cl][PF₆] (C2)

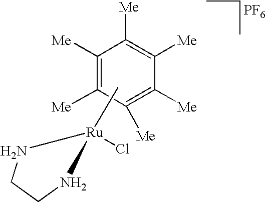

This complex was prepared in an analogous manner to compound C1 in Comparative Example 1 from [(η⁶-C₆(CH₃)₆)RuCl₂]₂. Yield of C2: 68%. Anal. Calcd. for C₁₄F₆H₁₂N₂ClPRu: C, 33.59; H, 4.43; N, 5.60 Found: C, 33.55; H, 4.57; N, 5.54

### Example 2

### Synthesis of [(η⁶-C₆(CH₃)₆)Ru(en)(pyridine)][PF₆]₂ (2)

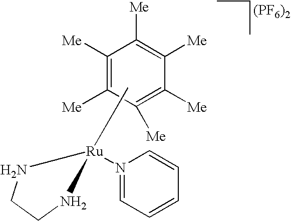

This complex was prepared by refluxing complex C2 (25.0 mg, 0.0496 mmol) and AgNO₃ (8.4 mg, 0.0494 mmol) in 2.5 mL of a 1:1 mixture of MeOH and H₂O for one hour. AgCl was removed by filtration. Pyridine (101 μl, 1.25 mmol) was added and the mixture was left overnight. The volume was reduced to ca. 1.5 mL by rotary evaporation and 100 mg of NH₄ PF₆ was added. The yellow precipitate was dissolved in acetone. The solution was then filtered and the acetone allowed to evaporate slowly, resulting in a microcrystalline, yellow product. Yield of 2: 19.3 mg (56%). Anal. Calcd for C₁₉F₁₂H₃₁N₃P₂Ru: C, 32.96; H, 4.51; N, 6.07. Found: C, 33.47; H, 4.50; N, 6.24.

### Example 3

### Synthesis of [(η⁶-C₆(CH₃)₆)Ru(en)(SCN)][PF₆]₂ (3)

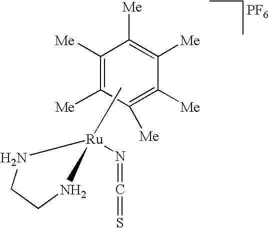

This complex was prepared by refluxing complex C2 (25.0 mg, 0.0496 mmol) and AgNO₃ (7.0 mg, 0.0412 mmol) in 2.5 mL of a 1:1 mixture of MeOH and H₂O for one hour. AgCl was removed by filtration. KSCN was added (243 mg, 2.50 mmol) and the solution stirred for one day. 150 mg of KPF₆ was added, and enough acetone was added to dissolve the resulting precipitate. Slow evaporation of the acetone yielded yellow crystals, which were suitable for X-ray crystallography studies. Yield: 6.9 mg (26%)

X-ray crystal structure determination yielded the result shown below, from which it can be seen that the isothiocyanate is bound via the nitrogen atom.

Crystal data and structure refinement for compound 3

X-ray data:

| | | |
|---|---|---|
| Crystal Data | | |
| Empirical formula | C15 H26 F6 N3 O P Ru S | |
| Formula weight | 542.49 | |
| Crystal system | Orthorhombic | |
| Space group | Pca21 | |
| Unit cell dimensions | a = 14.7411(12) Å | α = 90 deg. |
| | b = 9.0154(7) Å | β = 90 deg. |
| | c = 15.6070(12) Å | γ = 90 deg. |
| Volume | 2074.1(3) A³ | |
| Z | 4 | |
| Data Collection | | |
| Instrument | Bruker Smart Apex CCD | |
| Solution and Refinement | | |
| Solution | Patterson (Dirdif) | |
| R1 = 0.0619 [5064 data] | | |

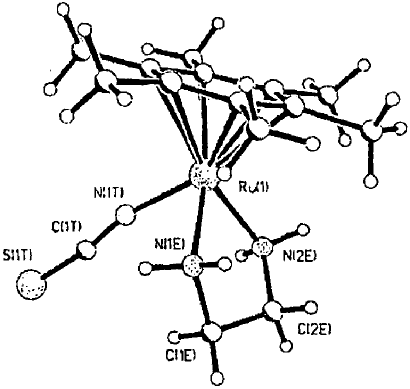

### Example 4

### Synthesis of [(η⁶-C₆(CH₃)₆)Ru(en)(SPh)][PF₆] (4)

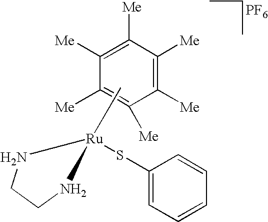

This complex was prepared by refluxing complex C2 (25.0 mg, 0.0496 mmol) and AgNO₃ (8.4 mg, 0.0494 mmol) in 2.5 mL of a 1:1 mixture of MeOH and H₂O for one hour. AgCl was removed by filtration. NaSPh was added (7.9 mg, 0.0595 mmol) and the solution was left overnight. 250 mg of NH₄ PF₆ was added, leading to an orange precipitate. Slow evaporation of the acetone solution of the precipitate led to a crystalline orange product and a yellow powder, both of which, by mass spectrometry, seemed to be the desired compound. Yield: 10.2 mg (36%). MS: m/z 433.0 for [M-PF₆]⁺ (Calc. 433.1).

### Example 5

### Synthesis of [(η⁶-C₆(CH₃)₆)Ru(en)N₃][PF₆] (5)

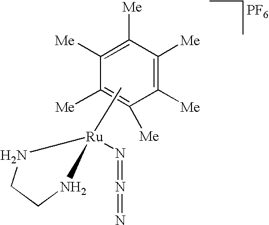

This complex was prepared by refluxing complex C2 (25.0 mg, 0.0496 mmol) and AgNO₃ (8.4 mg, 0.0494 mmol) in 2.5 mL of a 1:1 mixture of MeOH and H₂O for one hour. AgCl was removed by filtration. NaN₃ was added (163 mg, 2.51 mmol), dissolved by heating, and left overnight. NH₄ PF₆ (250 mg) was added, leading to a microcrystalline, yellow precipitate. Recrystallization of the precipitate from acetone gave to a yellow crystalline product. Yield of 5: 16.4 mg (65%). Anal. Calcd for C₁₄F₆H₂₆N₅PRu: C 32.94, H 5.13, N 13.72. Found: C, 32.32; H, 4.45; N, 12.63.

### Example 6

### Synthesis of [(η⁶-C₆(CH₃)₆)Ru(en)(3,5-dichloropyridine)][PF₆]₂ (6)

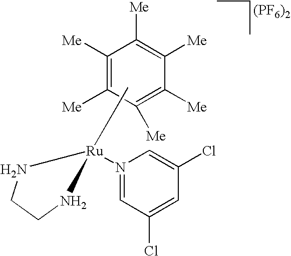

This complex was prepared in an analogous manner to compound 2 in Example 2. MS: m/z 616.0 for [6-PF₆]⁺ (Calc. 616.0)

### Example 7

### Synthesis of [(η⁶-C₆(CH₃)₆)Ru(en)(3,5-difluoropyridine)][PF₆]₂ (7)

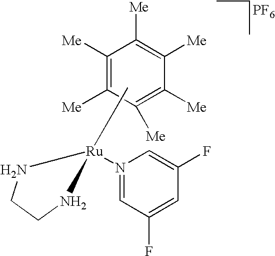

This complex was prepared in an analogous manner to compound 2 in Example 2. MS: m/z 583.9 for [7-PF₆]⁺ (Calc. 584.1)

### Example 8

### Synthesis of [(η⁶-C₆(CH₃)₆)Ru(en)(p-cyanopyridine)][PF₆]₂ (8)

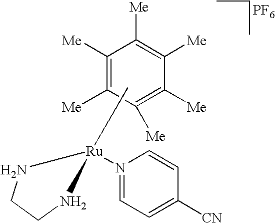

This complex was prepared in an analogous manner to compound 2 in Example 2. MS: m/z 572.9 for [8-PF₆]⁺ (Calc. 573.1)

X-ray data:

| | | |
|---|---|---|
| Crystal Data | | |
| Empirical formula | C20 H30 F12 N4 P2 Ru | |
| Formula weight | 717.49 | |
| Crystal system | Monoclinic | |
| Space group | P2(1)/n | |
| Unit cell dimensions | a = 8.6230(2) Å | α = 90° |
| | b = 34.7990(10) Å | β = 114.4360(10)° |
| | c = 9.8620(3) Å | γ = 90° |
| Volume | 2694.22(13) Å³ | |
| Z | 4 | |
| Data Collection | | |
| Absorption correction | SADABS | |
| Solution and Refinement | | |
| Solution | direct (SHELXS-97) | |
| Program used for | SHELXL-97 | |
| refinement | | |
| R1 = 0.0575 [4950 data] | | |

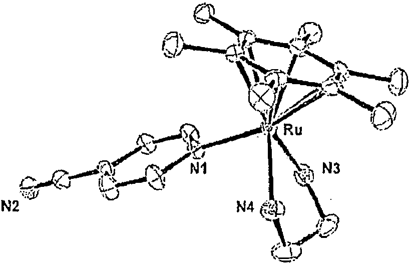

### Example 9

### Synthesis of [(η⁶-C₆(CH₃)₆)Ru(en)(3-methylpyridine)][PF₆]₂ (9)

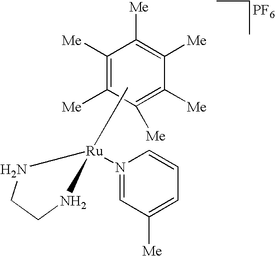

This complex was prepared in an analogous manner to compound 2 in Example 2. MS: m/z 562.1 for [9-PF₆]⁺ (Calc. 562.1)

X-ray data:

| | | |
|---|---|---|
| Crystal Data | | |
| Empirical formula | C20 H27 F12 N3 P2 Ru1 | |
| Formula weight | 700.45 | |
| Crystal system | Orthorhombic | |
| Space group | Pna21 | |
| Unit cell dimensions | a = 21.3199(6) Å | α = 90° |
| | b = 7.7155(2) Å | β = 90° |
| | c = 16.1809(5) Å | γ = 90° |
| Volume | 2661.66 Å³ | |
| Z | 4 | |
| Data Collection | | |
| Absorption correction | SADABS | |
| Solution and Refinement | | |
| Solution | direct (SHELXS-97) | |
| Program used for refinement | SHELXL-97 | |
| R1 = 0.0444 | | |

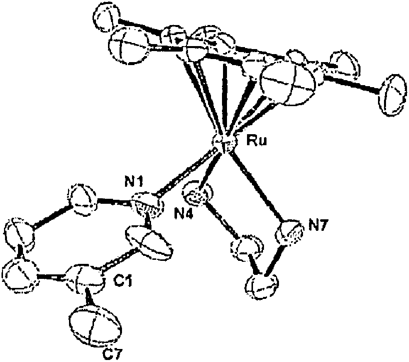

### Example 10

### Analysis of Compounds

Methods

Ultraviolet and Visible (UV-Vis) Spectroscopy: A Perkin-Elmer Lambda-16 UV-Vis spectrophotometer was used with 1-cm path-length quartz cuvettes (0.5 mL) and a PTP1 Peltier temperature controller. Spectra were processed using UVWinlab software for Windows' 95.

Kinetic Studies: Aliquots of stock solutions of the complexes to be tested (4-10 mM) in methanol were diluted to 500 μL with water, and the absorbance at selected wavelengths (determined by hydrolysis in an 19:1 mixture of water and methanol—see table 1 (λ)) was then recorded at 6 to 20 second intervals depending on the hydrolysis rate of each complex at 298 K. The hydrolysis rate constant k_{H2O} for each complex was determined by computer fit of the absorbance/time data for each complex to the first-order rate equation (eq. 1),
A=C₀+C₁e^{-kt} (1)
where C₀ and C₁ are computer-fitted constants, and A is the absorbance corresponding to time t, and the results are reported in table 1 as the half life (t_{1/2}).
Cytoxicity Studies

A2780 (1^{st} Method): A2780 cells were plated on day zero, and the complexes to be tested were added on day 3. The complex was removed on day 4 (i.e., 24 h cell exposure), and after growth in fresh medium in the absence of drug, the cells were counted on day 7. The complexes were stored in the dark at 277 K as a precaution against photochemical decomposition. The IC₅₀ (dose of compound required to cause 50% inhibition of cell growth) values are listed in Table 1.

A2780 (2^{nd} method) and A549: Cell line A2780 (human ovarian carcinoma, ECACC 93112519) was maintained in medium comprising RPMI-1640 (Sigma) with 5% Fetal Bovine Serum (Invitrogen), 2 mM L-Glutamine (Sigma) and 1% Penicillin/Streptomycin (Invitrogen), in T-75 flasks (Costar). Cells were passaged at approximately 75-90% confluence (1:8 dilution) using 0.25% Trypsin/EDTA (Invitrogen)

Cell Line A549 (human lung carcinoma, ECACC 86012804) was maintained in medium comprising DMEM (Sigma) with 10% Fetal Bovine Serum (Invitrogen), 2 mM L-Glutamine (Invitrogen) and 1% Penicillin/Streptomycin (Invitrogen), in T-75 flasks (Costar). Cells were passaged at approximately 70-90% confluence (1:8 dilution) using 0.25% Trypsin/EDTA (Invitrogen).

Both cell lines were incubated at 37° C., 5% CO₂, in high humidity.

A2780 carcinoma cells were seeded (150 μL) into 96 well plates (Nunc Maxisorp) at 5000 (±10%) cells per well and incubated at 37° C., 5% CO₂ in high humidity for 48 hours. A549 carcinoma cells were seeded (150 μL) into 96 well plates (Nunc Maxisorp) at 2000 (±10%) cells per well and incubated at 37° C., 5% CO₂ in high humidity for 24 hours.

The compounds to be tested were solubilised by sonication in DMSO (Fisher Scientific) to provide 20 mM solutions. Compounds were serially diluted with DMSO before diluting in cell culture medium to give concentrations four-fold greater than the final concentrations required in the assay. The dilutions of compound in culture medium were added to the cell plates (50 μL) in triplicates to achieve final concentrations of 100 μM, 50 μM, 10 μM, 5 μM, 1 μM and 0.1 μM. The final DMSO concentration in each well was 0.5% (v/v). The plates were incubated for 24 hours at 37° C., 5% CO₂, in high humidity

After 24 hours incubation, the cells were washed (200 μL) twice with sterile phosphate buffered Saline (Sigma) and the cell culture medium replenished (200 μL). Plates were incubated at 37° C., 5% CO₂, in high humidity for 96 hours. After the incubation surviving cells were fixed by the addition of 50% (w/v) Trichloroacetic acid (50 μL) and incubated at 4° C. for 1 hour. Plates were washed three times with excess tap water and air-dried. Cells were dyed by the addition of (100 μL) 0.4% sulforhodamine B (Sigma) solution to the plates followed by five washes (200 μl) with 1% acetic acid solution to remove excess dye before air-drying. Dye was re-solubilised in (200 μL) 10 mM Tris buffer (Fisher Scientific) and the absorbance of each well read at both 565 nm and 690 nm using a BMG Fluorostar microplate reader. The reading at 690 nm was subtracted from the 565 nm reading, and the IC₅₀ values determined by plotting the corrected absorbance value Vs. the compound concentration in the wells (XLfit version 4.0, ID Business Solutions Ltd). These are shown below in table 1

Results

The results of the above analyses are shown in table 1 below.

TABLE 1

| | | | IC₅₀ (μM) | | |
|---|---|---|---|---|---|
| | | | A2780 | A2780 | |
| Compound | λ (nm) | t_{1/2} (min) | (1^{st} method) | (2^{nd} method) | A549 |
| C1 | 260 | 5.0 | 8 | 6.5 | 11 |
| 1 | 270 | 367 | 4 | 14 | 8.5 |
| C2 | 254 | 0.44 | 9 | | |
| 2 | —^{a} | —^{a} | | | |
| 3 | | | | 24 | |
| 4 | —^{a} | —^{a} | 23 | 23 | 38 |
| 5 | 270 | 21.3 | 18 | 7.9 | |
| 6 | 270 | 537 | 23 | 18 | |
| 7 | 270 | 555 | | | |
| 8 | 270 | 43.9 | 6 | | |
| 9 | 254 | —^{a} | 50 | 50 | |

^{a}— no hydrolysis observed by UV-VIS

1. A ruthenium (II) compound of formula (I): 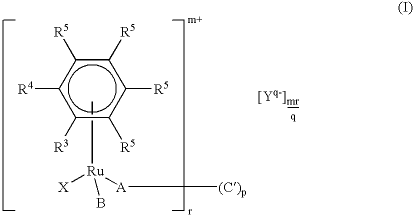
or a solvate or prodrug thereof, wherein:
R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is a neutral or negatively charged N- or S-donor ligand;
Y is a counterion;
m is 0 or 1;
q is 1, 2 or 3;
C′ is C1-12 alkylene bound to two A groups;
p is 0 or 1 and r is 1 when p is 0 and r is 2 when p is 1; and
A and B are each independently O-donor, N-donor or S-donor ligands, and may be connected to one another.

2. A compound according to claim 1, wherein X is selected from azide, isothiocyanate, and optionally substituted pyridine ligands.

3. A compound according to claim 1, wherein X is a thiolate ligand.

4. A compound according to claim 1 wherein A and B together represent NR^{N4}R^{N5}—(CR^{C1}R^{C2})n-NR^{N6}R^{N7}, wherein R^{C1} and R^{C2} are independently selected from 1-1 and C₁₋₄ alkyl, R^{N4}, R^{N5}, R^{N6} and R^{N7} are independently selected from 1-1 and C₁₋₄ alkyl, and n is an integer from 1 to 4.

5. A compound according to claim 4, wherein R¹⁴ and R¹⁵ are both hydrogen.

6. A compound according to claim 4, wherein n is 2.

7. A compound according to claim 4, wherein R^{N4}, R^{N5}, R^{N6} and R^{N7} are H.

8. A compound according to claim 1, wherein R¹ and R² together with the ring, to which they are attached form a saturated or unsaturated carbocyclic or heterocyclic group containing up to 3- to 8-membered carbocyclic or heterocyclic rings, wherein each carbocyclic or heterocyclic ring may be fused to one or more other carbocyclic or heterocyclic rings.

9. A compound according to claim 8, wherein R³, R⁴, R⁵ and R⁶ are H.

10. A compound according to claim 1, wherein R¹, R², R³, R⁴, R⁵ and R⁶ are independently selected from C₁₋₇ alkyl, C₅₋₂₀ aryl, C₃₋₂₀ heterocyclyl, halo, ester, amido, acyl, sulfo, sulfonamido, ether, thioether, azo and amino.

11. A compound according to claim 10, wherein R¹, R², R³, R⁴, R⁵ and R⁶ are independently selected from 1-1 and C₁₋₇ alkyl.

12. A compound according to claim 10, wherein at least four of R¹, R², R³, R⁴, R⁵ and R⁶ are hydrogen.

13. A composition comprising a compound according to claim 1, and a pharmaceutically acceptable carrier or diluent.

14. (canceled)

15. (canceled)

16. A method of treatment of a subject suffering from cancer, comprising administering to such a subject a therapeutically-effective amount of a compound according to claim 1.

17. A compound according to claim 2, wherein A and B together represent NRN4RN5-(CRC1RC2)n-NRN6RN7, wherein RC1 and RC2 are independently selected from H and C1-4 alkyl, RN4, RN5, RN6 and RN7 are independently selected from H and C1-4 alkyl, and n is an integer from 1 to 4.

18. A compound according to claim 2, wherein A and B together represent NR^{N4}R^{N5}—(CR^{C1}R^{C2})n-NR^{N6}R^{N7}, wherein R^{C1} and R^{C2} are independently, selected from H and C₁₋₄ alkyl, R^{N4}, R^{N5}, R^{N6} and R^{N7} are independently selected from H and C₁₋₄ alkyl, and n is an integer from 1 to 4.

19. A compound according to claim 5, wherein R^{N4}, R^{N5}, R^{N6} and R^{N7} are H.

20. A compound according to claim 6, wherein R^{N4}, R^{N5}, R^{N6} and R^{N7} are H.

21. A compound according to claim 1 wherein at least four of R¹, R², R³, R⁴, R⁵ and R⁶ are hydrogen.
